(19) United States
(12) Patent Application Publication
(10) Pub. No.: US 2026/0290607 A1
(43) Pub. Date: Sep. 24, 2026

(54) MULTI-MODALITY DEEP LEARNING FOR VENTRICULAR ARRHYTHMIA RISK PREDICTION

(71) Applicant: THE JOHNS HOPKINS UNIVERSITY, Baltimore, MD (US)

(72) Inventors: Changxin LAI, Baltimore, MD (US); Minglang YIN, Towson, MD (US); Natalia A. TRAYANOVA, Baltimore, MD (US)

(21) Appl. No.: 19/484,479

(22) PCT Filed: May 16, 2024
(86) PCT No.: PCT/US2024/029582

§ 371 Date: Nov. 14, 2025

Related U.S. Application Data
(60) Provisional application No. 63/502,699, filed on May 17, 2023.

(51) Int. Cl. G16H 50/30; G06T 7/00; G16H 10/60

(52) U.S. Cl. CPC G16H 50/30; G06T 7/0012; G16H 10/60; G06T 2207/10088; G06T 2207/20084; G06T 2207/30048

(57) ABSTRACT

Machine learning techniques for ventricular arrhythmia risk prediction for a hypertrophic cardiomyopathy patient are presented. The techniques include obtaining a patient late gadolinium enhancement cardiac MRI (LGE-MRI) image of at least the patient's left ventricle; obtaining patient electronic health records data of the patient; providing the patient data to a trained machine learning system that include: an image branch trained using a training corpus of LGE-MRI images, an electronic health records data branch trained using a training corpus of electronic health records data, and a multi-modality fusion component; obtaining, from the trained machine learning system, ventricular arrhythmia prediction data; and providing a ventricular arrhythmia prediction based on the ventricular arrhythmia prediction data.

### CROSS-REFERENCE TO RELATED APPLICATIONS

This application is the national stage entry of International Patent Application No. PCT/US2024/029582, filed on May 16, 2024, and published as WO 2024/238728 A2 on Nov. 21, 2024, which claims the benefit of U.S. Provisional Patent Application No. 63/502,699, entitled, “Multi-Modal Deep Learning Method for Ventricular Arrhythmia Risk Prediction in Hypertrophic Cardiomyopathy Patients,” filed May 17, 2023, which are hereby incorporated by reference herein in their entireties.

### GOVERNMENT SUPPORT

This invention was made with government support under grant HL142496 awarded by the National Institutes of Health. The government has certain rights in this invention.

### FIELD

This disclosure relates generally to deep learning for ventricular arrhythmia risk prediction, e.g., in hypertrophic cardiomyopathy patients.

### BACKGROUND

Sudden cardiac death (SCD) continues to be a major public health problem worldwide, with an incident of 50 to 100 per 100,000 population every year in the Europe and North America, accounting for 15-20% of all deaths. Most cardiac arrests occur outside of hospital and result in poor outcomes, with an overall survival rate less than 5%. Ventricular arrhythmias (VA) are often the underlying mechanisms for SCD. Implantable cardioverter defibrillator (ICD) can effectively end most episodes of VA and reduce the total mortality in high-risk groups of patients who have not yet had VA. However, ICD recipients are also exposed to various risks including device implantation, generator replacement, and the potential drawbacks of living with ICD.

The current paradigm of SCD risk stratification remains suboptimal. Most ICD recipients do not experience meaningful health benefits from the device, and, meanwhile, some patients who truly need it are not well protected. The most common clinical criteria—left ventricle ejection fraction (LVEF)<30-35%—or other risk factors outlined in clinical guidelines only provide a limited view of patients' conditions, and some of the factors, especially the imaging features, require expert interpretation and are thus prone to high inter-reader variability. The limitations altogether lead to inconsistent sensitivity and specificity in previous validation studies. Accurate risk stratification for guiding appropriate ICD implantation is important for preventing SCD and improving healthcare.

SCD risk stratification is even more challenging for patients with non-ischemic cardiomyopathies (NICM), which features a diverse range of etiologies and mechanisms. Hypertrophic cardiomyopathy (HCM), for instance, is one type of NICM and the most prevalent inherited cardiac disease. HCM affects 1 in every 200 to 500 individuals and is a primary cause of SCD in the young population. Previous efforts to refine risk stratification for HCM patients have various limitations. Most work has focused on either identifying additional risk factors from new perspectives, such as electrocardiogram (ECG) changes and genetic variants, or on automating image interpretation to improve measurement accuracy and consistency. Despite the improvements, these models fail to integrate multiple relevant factors.

### SUMMARY

According to various embodiments, a machine learning method for ventricular arrhythmia risk prediction for a hypertrophic cardiomyopathy patient is presented. The method includes: obtaining a patient late gadolinium enhancement cardiac MRI (LGE-MRI) image of at least the patient's left ventricle; obtaining patient electronic health records data of the patient; providing patient data to a trained machine learning system, wherein the patient data comprises the patient LGE-MRI image and the patient electronic health records data, wherein the trained machine learning system comprises: an image branch trained using a training corpus of LGE-MRI images, an electronic health records data branch trained using a training corpus of electronic health records data, and a multi-modality fusion component, wherein the multi-modality fusion component is coupled to at least the image branch and the electronic health records data branch, and wherein the providing comprises inputting the patient LGE-MRI image to the image branch and inputting at least some of the patient electronic health records data to the electronic health records data branch; obtaining, from the trained machine learning system, ventricular arrhythmia prediction data; and providing a ventricular arrhythmia prediction based on the ventricular arrhythmia prediction data.

Various optional features of the above method embodiments include the following. The method may include: determining that the ventricular arrhythmia prediction exceeds a predetermined threshold for a numeric ventricular arrhythmia prediction, or determining that the ventricular arrhythmia prediction is in a predetermined category for a categorical arrhythmia prediction; and treating the patient with an implantable cardioverter defibrillator in response to the determining. The patient electronic health records data may include patient cardiac imaging report data of the patient, the trained machine learning system may further include a cardiac imaging report data branch trained using a training corpus of cardiac imaging report data, the providing may further include inputting the cardiac imaging report data to the cardiac imaging report data branch, and the multi-modality fusion component may be further coupled to the cardiac imaging report data branch. The patient electronic health records data may include: patient demographics data and patient medical records data, and the patient cardiac imaging report data may include patient echocardiogram data and patient cardiac MRI data. The patient electronic health records data may include: patient demographics data, patient medical records data, patient echocardiogram data, and patient cardiac MRI data. The method may include automatically identifying a region of interest that tightly contains a left ventricular myocardium of the patient LGE-MRI image using a neural network. The ventricular arrhythmia prediction data may represent a probability of sudden cardiac death due to arrhythmia during a specified timeframe. The image branch may include a three dimensional vision transformer. The image branch may include a first neural network, and the electronic health records branch may include a second neural network. The multi-modality fusion branch may include a transformer.

According to various embodiments, a machine learning system for ventricular arrhythmia risk prediction for a hypertrophic cardiomyopathy patient is presented. The system includes: a non-transitory computer readable medium comprising instructions; and at least one electronic processor that executes the instructions to perform operations comprising: obtaining a patient late gadolinium enhancement cardiac MRI (LGE-MRI) image of at least the patient's left ventricle; obtaining patient electronic health records data of the patient; providing patient data to a trained machine learning system, wherein the patient data comprises the patient LGE-MRI image and the patient electronic health records data, wherein the trained machine learning system comprises: an image branch trained using a training corpus of LGE-MRI images, an electronic health records data branch trained using a training corpus of electronic health records data, and a multi-modality fusion component, wherein the multi-modality fusion component is coupled to at least the image branch and the electronic health records data branch, and wherein the providing comprises inputting the patient LGE-MRI image to the image branch and inputting at least some of the patient electronic health records data to the electronic health records data branch; obtaining, from the trained machine learning system, ventricular arrhythmia prediction data; and providing a ventricular arrhythmia prediction based on the ventricular arrhythmia prediction data.

Various optional features of the above system embodiments include the following. The operations may further include: determining that the ventricular arrhythmia prediction exceeds a predetermined threshold for a numeric ventricular arrhythmia prediction, or determining that the ventricular arrhythmia prediction is in a predetermined category for a categorical arrhythmia prediction, wherein the patient is treated with an implantable cardioverter defibrillator in response to the determining. The patient electronic health records data may include patient cardiac imaging report data of the patient, the trained machine learning system may further include a cardiac imaging report data branch trained using a training corpus of cardiac imaging report data, the providing may further include inputting the cardiac imaging report data to the cardiac imaging report data branch, and the multi-modality fusion component may be further coupled to the cardiac imaging report data branch. The patient electronic health records data may include: patient demographics data and patient medical records data, and the patient cardiac imaging report data may include patient echocardiogram data and patient cardiac MRI data. The patient electronic health records data may include: patient demographics data, patient medical records data, patient echocardiogram data, and patient cardiac MRI data. The operations may further include automatically identifying a region of interest that tightly contains a left ventricular myocardium of the patient LGE-MRI image using a neural network. The ventricular arrhythmia prediction data may represent a probability of sudden cardiac death due to arrhythmia during a specified timeframe. The image branch may include a three dimensional vision transformer. The image branch may include a first neural network, and the electronic health records branch may include a second neural network. The multi-modality fusion branch may include a transformer.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 is a schematic diagram of a tri-channel machine learning system for ventricular arrhythmia risk prediction for a hypertrophic cardiomyopathy patient, according to a first reduction to practice;

FIG. 2 depicts receiver operating characteristics curves comparing the tri-channel reduction to practice to both clinical guidelines and to its constituent sub-networks, via both internal cross-validation using a first cohort and external validation using a second cohort;

FIG. 3 illustrates interpretability of the tri-channel reduction to practice based on attention to various regions of LGE-MRI images;

FIG. 4 is a schematic diagram of a bi-channel machine learning system for ventricular arrhythmia risk prediction for a hypertrophic cardiomyopathy patient, according to a second reduction to practice;

FIG. 5 depicts receiver operating characteristics curves comparing the bi-channel reduction to practice to both clinical guidelines and to its constituent sub-networks; and

FIG. 6 is a flow diagram for a machine learning method for ventricular arrhythmia risk prediction for a hypertrophic cardiomyopathy patient, according to various embodiments.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention and it is to be understood that other examples may be utilized and that changes may be made without departing from the scope of the invention. The following description is, therefore, merely exemplary.

### I. Introduction

Various embodiments include a scalable multi-modality deep learning (DL) method for ventricular arrhythmia risk stratification, e.g., in HCM patients. The DL method can automatically analyze multiple modalities of input data, e.g., images, including raw late gadolinium enhancement cardiac magnetic resonance (LGE-MRI) images, and clinical covariates from electronic health records (EHR). According to some embodiments, the clinical covariates from EHR data may include cardiac imaging report (CIR) data. According to some embodiments, EHR data including CIR data is passed to a single channel in a trained machine learning system; according to other embodiments, the CIR data is passed to a channel different from a channel that receives other EHR data. Various embodiments may use dedicated neural networks to extract features from raw LGE-MRI images (e.g., based on pixel intensity of the raw images) and EHR data, the latter of which may be split and passed to two or more channels, e.g., CIR data and other EHR data, fuse the multi-modality knowledge, and output a personalized risk score for VA.

According to various embodiments, the personalized risk score for VA is in the range between 0 and 1, representing the patient's risk for VA, where 0 means no risk and 1 means deterministically high risk. The risk score may represent a probability of VA occurring within a specified timeframe, e.g., a specified number of years, such as two years or three years.

When the predicted risk score is higher than a decision threshold, the patient may be considered at high risk for VA and may be recommended to have implantable cardioverter defibrillator (ICD) deployment. ICD is used as the primary prevention of life-threatening VA in HCM patients, but it carries risk of potential complications and morbidity. Because reductions to practice have demonstrated higher accuracy in VA risk stratification than current clinical guidelines, an output risk score over the decision threshold followed by an ICD recommendation or deployment represents an improvement to HCM patient care by guiding this life-saving treatment to those who need it most.

According to some embodiments, training and patient LGE-MRI images undergo preprocessing that use a neural network to identify a region of interest in the image that tightly contains the left ventricle. Such preprocessing includes removing other areas of the LGE-MRI image so that the deep learning algorithm learns and predicts on raw pixel intensity in the ventricular walls only. According to some embodiments, there is no human interference in deciding what is the actual wall or what is fibrosis in it, as the preprocessing uses learned segmentation.

Reductions to practice have outperformed the latest clinical criteria outlined by ACC/AHA and ESC in predicting VA risk. The reductions to practice have also maintained excellent performance in different subgroups of patients, including gender, age, and race. The robustness of these results suggests that an embodiment can provide accurate risk stratification for a wide range of patients, e.g., HCM patients, regardless of demographic or clinical factors. Further, the interpretability of some embodiments allows for identification of specific regions from raw LGE-IRM images and covariates from EHR and/or cardiac imaging report (CIR) data that contributed to the personalized risk score for VA. With the accuracy, fairness, and transparency in identifying patients at high risk of VA, various embodiments may help improve patient care and guide life-saving treatment to those who need it most.

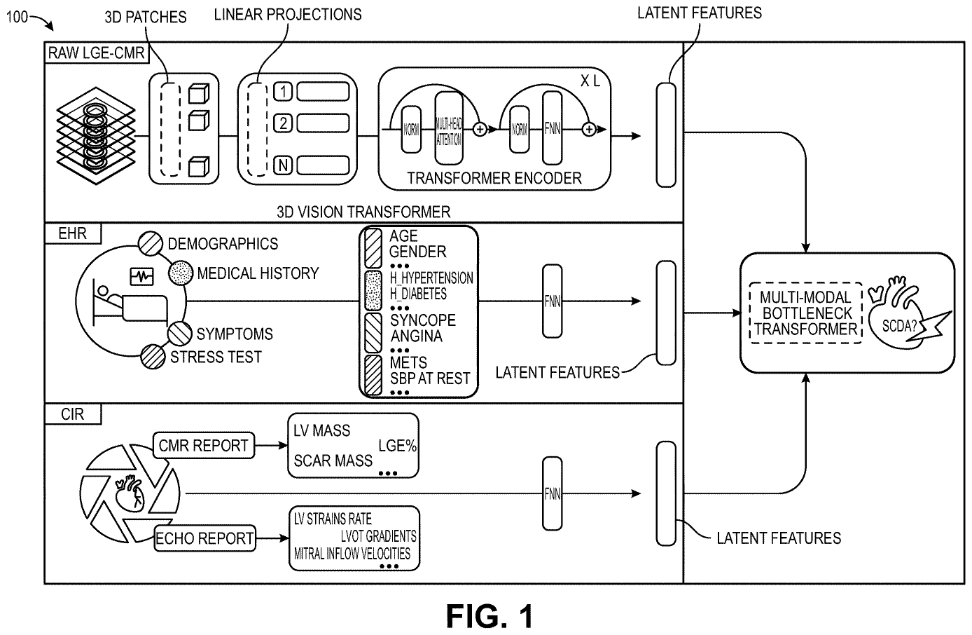

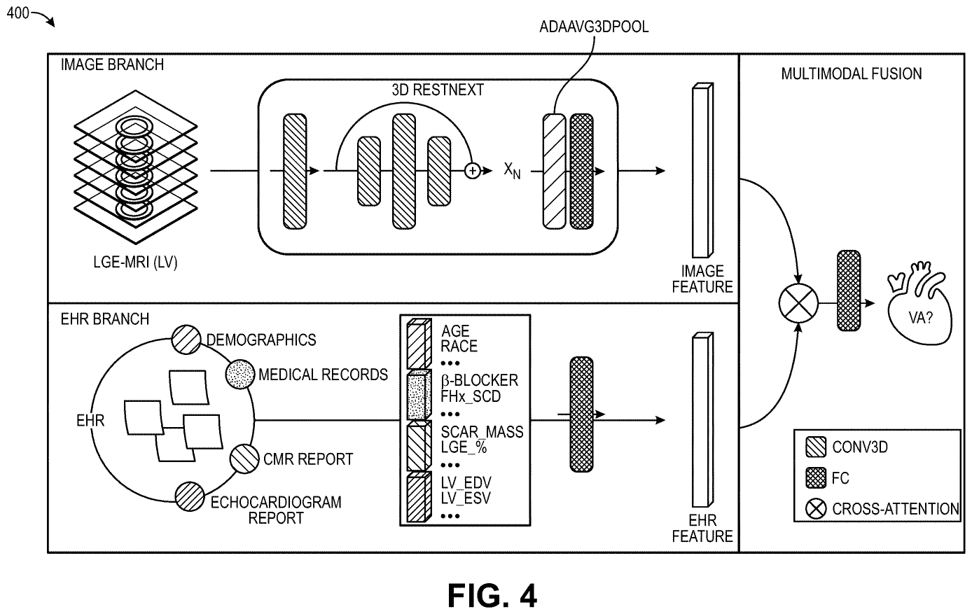

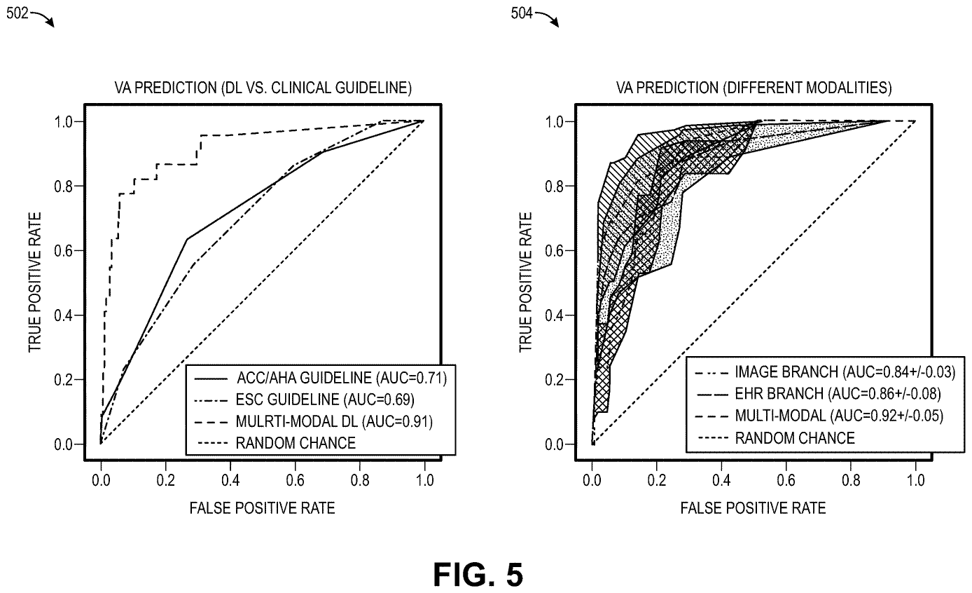

This description proceeds to describe two non-limiting example reductions to practice. A tri-channel reduction to practice is shown and described herein in Section II reference to FIGS. 1, 2, and 3. A bi-channel reduction to practice is shown and described herein in Section III in reference to FIGS. 4 and 5.

### II. Tri-Channel Reduction to Practice

This section presents a reduction to practice that predicts and interprets the risk of arrhythmic sudden cardiac death (SCDA) in patients using bi-modality medical data and evaluated in HCM patient cohorts. The tri-channel reduction to practice employs a transformer-based neural network to analyze data from electric health records (EHR), echocardiogram and LGE-MRI report data, as well as raw LGE-MRI images, which reveal disease-induced myocardial fibrosis. The tri-channel reduction to practice includes three unimodality sub-networks, or paths, for extracting modality-specific features, and a multi-modality fusion module to fuse knowledge and distill holistic representations for prognostic SCDA risk stratification. The high performance, fairness, and generalizability of the tri-channel reduction to practice are demonstrated in interval cross-validations and an independent external test. The tri-channel reduction to practice also uses attention- and attribution-based interpretability to improve decision transparency. With reliable performance and interpretability as demonstrated by the tri-channel reduction to practice, some embodiments have the potential to revolutionize SCDA risk stratification in HCM, fostering a collaborative, AI-assisted decision-making paradigm that could significantly improve precision care.

An outline of the description of the tri-channel reduction to practice as presented in this section follows. Two HCM patient cohorts from geographically separate communities were used to develop and validate the tri-channel reduction to practice. A first “internal” cohort of 553 patients from the was used to construct a 5-fold cross-validation study, on which the tri-channel reduction to practice was developed and internally validated. To evaluate the generalizability and cross-healthcare-system reliability of the tri-channel reduction to practice, after the development of the model using the first cohort, it was further tested on an independent second “external” cohort of 284 HCM patients from a different medical facility than that of the first cohort. To establish a baseline level of clinical risk prediction accuracy for this cohort, widely used clinical tools are applied, including the 2020 ACC/AHA guidelines for HCM, the 2023 ESC guidelines for cardiomyopathies, and the HCM Risk-SCD calculator. During the internal and external validations, the performance of the tri-channel reduction to practice was compared with the clinical tools to demonstrate the improvements offered by multi-modality AI according to various embodiments, and the tri-channel reduction to practice was compared with the individual unimodality sub-networks of the tri-channel reduction to practice to delineate the effects of including multiple data modalities. The performance of the different models is also evaluated in different sex- and age-subgroups to analyze the fairness of the tri-channel reduction to practice herein. Furthermore, using the interpretability and predictions offered by the tri-channel reduction to practice, the predictions made and the knowledge learned from each data modality are analyzed herein.

The internal cohort included patients who visited a first medical facility during 2005 to 2015 and were diagnosed with HCM. The patients were followed for a mean duration of 2.86 years; therefore, the risk score output from the tri-channel reduction to practice reflected a patient's risk for SCDA, explained presently, in this timeframe. The primary endpoint SCDA for the first cohort is defined as sustained ventricular tachycardia (VT) (ventricular rate 130 beats/min/≥30 seconds duration) or ventricular fibrillation (VF), resulting in defibrillator shocks or anti-tachycardia pacing. Among the 553 patients included in the first cohort, 19 patients (3%) experienced SCDA during the follow-up.

The external cohort included patients who visited second medical facility. The primary endpoint SCDA for the second cohort is defined for the purpose of validation as device shock, appropriate interventions, or out-of-hospital cardiac arrest. Among the 284 patients included in the second cohort, 25 patients (9%) experienced SCDA during the follow-up.

FIG. 1 is a schematic diagram of a tri-channel machine learning system 100 for ventricular arrhythmia risk prediction for a hypertrophic cardiomyopathy patient, according to the first reduction to practice. The system 100 includes dedicated unimodality sub-network branches designed for three different inputs, including raw LGE-MRI images, clinical covariates from EHR, and measurements from cardiac imaging reports (CIR). Each unimodality branch extracted respective features from the respective input data. The system 100 also includes a multi-modality fusion component to synergistically fuse the extracted features and generate a corresponding output. The target outcomes, used as labels for the training data, were the event type (SCDA or non-SCDA).

The multi-modality inputs to the tri-channel reduction to practice include image data (e.g., raw LGE-MRI images) and numeric/Boolean parameter data (e.g., clinical data from electronic health records (EHR) and measurements from cardiac imaging reports (CIR)). The CIR data includes numeric or Boolean parameters determined using echocardiogram and cardiac magnetic resonance imaging. Table 1 presents EHR variables, and Table 2 presents CIR variables, as used by the tri-channel reduction to practice. In Table 1 and Table 2, the left column shows the variable name, and the right column describes the respective variable, its units where applicable, and its data type (e.g., continuous or nominal). In addition, in Table 2, the left column indicates whether values for the respective CIR variable were obtained by echocardiogram (ECHO) or LGE-MRI (CMR).

TABLE 1
EHR Covariates

| | |
|---|---|
| Age | (years); Continuous |
| BMI | Body mass index (kg/m\hat{ }2); Continuous |
| DBP_rest | Stress test: diastolic blood pressure at rest (mmHg); Continuous |
| DBP_stress | Stress test: diastolic blood pressure at peak exercise (mmHg); |
| | Continuous |
| HR_recovery_60s | Stress test: heart rate at 1 min post-exercise (bpm); Continuous |
| HR_rest | Stress test: heart rate at rest, pre-exercise (bpm); Continuous |
| HR_stress | Stress test: heart rate at peak exercise (bpm); Continuous |
| METS | Stress test: metabolic equivalents; Continuous |
| Percentage_of_MaxHr | Stress test: peak heart rate achieved, expressed as % of maximum |
| | predicted heart rate (%); Continuous |
| SBP_rest | Stress test: systolic blood pressure at rest (mmHg); Continuous |
| SBP_stress | Stress test: systolic blood pressure at peak exercise (mmHg); |
| | Continuous |
| height_m | Height (m); Continuous |
| weight_kg | Weight (kg); Continuous |
| excl_myect | Patient had myectomy before they came to JHU; Nominal |
| GENDER | Sex; Nominal |
| ANGINA | Symptoms: angina; Nominal |
| DYSPNEA at | Symptoms: dyspnea on exertion; Nominal |
| exertion | |
| DIZZINESS | Symptoms: dizziness; Nominal |
| PRESYNCOPE | Symptoms: presyncope; Nominal |
| SYNCOPE | Symptoms: syncope; Nominal |
| H_DM | History of diabetes; Nominal |
| Smoking | Current or previous smoker; Nominal |
| H_HTN | History of hypertension; Nominal |
| H_DL | History of dyslipidemia; Nominal |
| H_stroke | History of stroke; Nominal |
| H_CAD | History of obstructive CAD; Nominal |
| H_MI | History of myocardial infarction; Nominal |
| FHx_HCM | Family history of HCM; Nominal |
| ABPR | Stress test: abnormal blood pressure response during exercise; |
| | Nominal |
| FHx_SCD | Family history of sudden cardiac death; Nominal |
| H_Afib | History of atrial fibrillation; Nominal |
| H_NSVT | History of non-sustained ventricular tachycardia; Nominal |
| HF | Heart failure; Nominal |
| MI | Myocardial infarction; Nominal |
| Race_0 | Race: other; Nominal |
| Race_1 | Race: white: Nominal |
| Race_2 | Race: African American; Nominal |
| NYHA_1 | New York Heart Association functional classification: class 1; |
| | Nominal |
| NYHA_2 | New York Heart Association functional classification: class 2; |
| | Nominal |
| NYHA_3 | New York Heart Association functional classification: class 3; |
| | Nominal |

TABLE 2
CIR Covariates

| | |
|---|---|
| Apex | ECHO: apical thickness; Continuous |
| EA | ECHO: left ventricle diastolic function: E/A; Continuous |
| EE | ECHO: left ventricle diastolic function: E/e′; Continuous |
| EF | ECHO: left ventricle ejection fraction (%); Continuous |
| Global_SR_E | ECHO: left ventricle global longitudinal early diastolic strain rate, |
| | SR_E, (1/s); Continuous |
| Global_SR_S | ECHO: left ventricle global longitudinal systolic strain rate, SR_S |
| | (1/s); Continuous |
| Global_S_G | ECHO: left ventricle strain, global longitudinal systolic strain, LV- |
| | GLS (%); Continuous |
| IVS_PW | ECHO: interventricular septum/posterior wall thickness ratio; |
| | Continuous |
| IVS_max | ECHO: maximum LV interventricular septum thickness (mm); |
| | Continuous |
| LA | ECHO: left atrial diameter (mm); Continuous |
| LGE_% | CMR: late gadolinium enhancement percentage (%); Continuous |
| LVEDV | ECHO: LV end-systolic volume (ml); Continuous |
| LVESV | ECHO: LV end-diastolic volume (ml); Continuous |
| LVOT_Rest | ECHO: LV outflow tract gradient at rest (mmHg); Continuous |
| LVOT_Stress | ECHO: LV outflow tract gradient at peak stress (mmHg); |
| | Continuous |
| MV_A | ECHO: early mitral inflow velocity; Continuous |
| MV_DecT | ECHO: deceleration time of early mitral inflow (ms); Continuous |
| MV_E | ECHO: mitral inflow velocity during atrial systole; Continuous |
| PW | ECHO: LV posterior wall thickness (mm); Continuous |
| Scar_tissue_mass_g | CMR: LV scar tissue mass (g); Continuous |
| Total_myocardial_mass_g | CMR: LV total myocardium mass (g); Continuous |
| SAM | ECHO: systolic anterior motion of mitral valve (SAM); Nominal |
| IVS_3 | ECHO: IVS ≥3 cm; Nominal |
| LGE | CMR: late gadolinium enhancement presence; Nominal |
| HCMtype_0 | ECHO: non-obstructive HCM; Nominal |
| HCMtype_1 | ECHO: labile HCM; Nominal |
| HCMtype_2 | ECHO: Obstructive HCM; Nominal |

As shown in Tables 1 and 2, the EHR and CIR inputs both contain clinical features reflecting different aspects of the disease. The EHR input has 40 features accounting for patients' demographics, medical history, symptoms, and stress test results. The CIR input has 27 features derived from the echocardiogram reports and LGE-MRI radiology reports, which measure the cardiac functions and geometries with expert-defined markers. Note that the CIR input does not include cardiac images themselves; rather, the CIR input includes numeric and Boolean measurements derived from cardiac images. Separate feed-forward neural networks (FNN) are adopted in the system 100 for analyzing inputs from EHR and CIR, respectively.

The image sub-network takes raw LGE-MRI images as inputs. The images visualize the 3D geometries of patients' left ventricles (LV), as well as their disease-induced fibrosis in the myocardium, visible as contrast-enhanced regions, which mechanistically creates substrates for VA and leads to SCDA. A customized 3D Vision Transformer (3D-ViT) is adopted to analyze the raw LGE-MRI images and extract modality-specific knowledge. Compared with 2D or 2.5D models, the inherent 3D design preserves the full spatial information and enables the model to correctly learn correlations between LGE-MRI images and propensity for SCDA.

In the pre-processing of the LGE-MRI images, first the left ventricle (LV) region of interest is obtained, using a previously developed and validated deep learning method. In brief, this method uses a cascaded convolutional neural network that produces LV myocardium segmentations via three sub-networks: a U-net with residual connections trained to identify the entire region of interest; a U-net with residual connections trained to delineate the myocardium wall; and an encoder-decoder tasked with correcting anatomical inaccuracies that may have resulted in the segmentation. In this context, anatomical correctness is defined via a list of pass/fail rules (for example, no holes in the myocardium, circularity threshold and no disconnected components). Once each patient's LGE-MRI 2D slices are segmented via this method, all pixels outside the LV are zeroed out, and the pixels within the LV are linearly scaled by multiplication with half the inverse of the median blood pool pixel intensity in each slice. Finally, the processed slices are stacked and interpolated to a regular 96×96×20 grid with voxel dimensions 4.0×4.0×6.3 mm. The pre-processing steps are intended to exclude non-relevant background information and standardize the LGE-MRI image volume for consistent analysis across all patients.

For the system 100, the EHR and CIR data are structured as tabular data. The features included in the analysis were ensured to have less than 40% missing values originally, and the missing values were imputed using multivariate imputation by chained equations (MICE). MICE is a fully conditional specification approach that models each feature with missing values as a function of all other features iteratively. After imputation, the EHR and CIR data were standardized via Z-score, which involves subtracting the mean and dividing by the standard deviation of each feature.

The modality-specific sub-networks of the tri-channel reduction to practice are described in detail presently. As described, the tri-channel reduction to practice uses a mid-fusion paradigm, which uses modality-specific sub-networks to specialize in learning and extracting unimodality patterns, before the multi-modality information is fused. Three unimodality sub-networks are included in the tri-channel reduction to practice, each operating on a specific input data type: a 3D vision transformer (3D-ViT) for raw LGE-MRI images, a feedforward neural network (FNN) for EHR data, and an FNN for CIR data.

In the image (CMR) branch, the LGE-MRI image vector embeddings z are obtained by firstly creating n non-overlapping 3D image patches xᵢ ϵR^{16×16×4} from the original 3D image X ϵR^{96×96×20}, obtaining linear projections of the flattened image patches, appending a classification token z_{cls} (CLS-token), and adding positional embeddings:

$${z}_{CMR} = g ( x ; E . {z}_{cls} ) = [ {z}_{cls} , {Ex}_{1} , {Ex}_{2} , … , {Ex}_{n} ] + p \qquad ( 1 )$$

In Equation (1), E is a linear projection mapping each flattened image patch to R^{d}, with dimension d being a hyperparameter of the model, and pϵR^{(n+1)×d} is a learned positional embedding added to the embeddings to retain positional information. Learnable positional embeddings are employed in the ViT model here.

The image vector embeddings z are then processed by a sequence of L_{ViT} transformer encoder blocks. Each transformer encoder block, z^{l+1}=Transformer(z^{l}; θ_{ViT}), includes two sub-modules: (1) a multi-head self-attention (MSA) module, and (2) a fully connected FNN consisting of two linear transformations with a non-linear activation function in between. Each of these two sub-modules has a residual connection around it and a preceding normalization layer.

$${v}^{l} = MSA ( LN ( {z}^{l} ) ) + {z}^{l} \qquad ( 2 )$$

$${z}^{l + 1} = FNN ( LN ( {v}^{l} ) ) + {v}^{l} \qquad ( 3 )$$

In Equations (2) and (3), the MSA operation computes dot-product attention where the queries, keys and values are all linear projections of the same tensor, MSA(X)=Attention(W^{Q}X, W^{K}X, W^{V}X), and LN is the layer normalization operation.

After the final transformer encoder block, the linear mapping of the CLA-token as extracted CMR knowledge \tilde{z}_{CMR} are taken:

$${z}_{CMR}^{L + 1} = [ {z}_{cls}^{L + 1} , {z}_{1}^{L + 1} , {z}_{2}^{L + 1} , … , {z}_{n}^{L + 1} ] = {Transformer}^{{L}_{ViT}} ( {z}_{CMR} ; {θ}_{ViT} ) \qquad ( 4 )$$

$${\tilde{z}}_{CMR} = LN ( {z}_{cls}^{L + 1} \cdot W ) \qquad ( 5 )$$

In the EHR and CIR branches, processed EHR and CIR data are converted to vectors z_{EHR},z_{CIR} fed into specific fully connected FNNs. The FNNs each include multiple linear transformation layers, with each layer also succeeded by a normalization layer, a non-linear activation function, and a drop-out layer. The latent vectors \tilde{z}_{EHR} and \tilde{z}_{CIR} representing the EHR and CIR knowledge are extracted hereby.

$${\tilde{z}}_{EHR} = FNN ( {z}_{EHR} ) \qquad ( 6 )$$

$${\tilde{z}}_{CIR} = FNN ( {z}_{CIR} ) \qquad ( 7 )$$

Fusion of the unimodality branches of the tri-channel reduction to practice is described presently. Following the unimodality knowledge extraction from CMR, EHR, and CIR subnetworks, a Multi-modal Bottleneck Transformer (MBT) is used to fuse the knowledge and learn the cross-modality interactions. MBT has light computational cost. The idea of MBT is to restrict cross-modality attention flow between latent vectors, and it achieves this through tight fusion bottlenecks, which force the model to collect and condense the most relevant inputs in each modality, sharing only necessary information with other modalities. In every block of MBT, every unimodal latent vector, \tilde{z}ₘ, mϵ{CMR, EHR, CIR}, is concatenated with a shared small set of bottleneck fusion tokens \tilde{z}_{fsn}, and goes through a self-attention transformer; after the unimodal latent vectors are updated separately, the temporary bottleneck tokens from each update, \tilde{z}_{fsn}_{ₘ}, are then averaged to get the new share bottleneck tokens.

Formally, for layer l, the MBT computation

$$[ {\tilde{z}}_{m}^{l + 1} ] [ {\tilde{z}}_{{fsn}_{m}}^{l + 1} ] = Transformer ( [ {\tilde{z}}_{m}^{l} ] [ {\tilde{z}}_{fsn}^{l} ] ; {θ}_{MBT , m} ) \qquad ( 8 )$$

$${\tilde{z}}_{fsn}^{l + 1} = Avg ( {\hat{z}}_{{fsn}_{m}}^{l + 1} ) \qquad ( 9 )$$

is as follows:

$${\tilde{z}}_{CMR}^{l + 1} , {\tilde{z}}_{EHR}^{l + 1} , {\tilde{z}}_{CIR}^{l + 1} , {z}_{fsn}^{l + 1} = MBT ( {\tilde{z}}_{CMR}^{l} , {\tilde{z}}_{EHR}^{l} , {\tilde{z}}_{CIR}^{l} , {z}_{fsn}^{l} ; {θ}_{MBT} )$$

After a sequence of L_{MBT} blocks of MBT, a fully connected layer takes the concatenation of all the latent vectors and provides an output r, which represents the predicted SCDA risk score.

$${\tilde{z}}_{CMR}^{{L}_{MBT} + 1} , {\tilde{z}}_{EHR}^{{L}_{MBT} + 1} , {\tilde{z}}_{CIR}^{{L}_{MBT} + 1} , {z}_{fsn}^{{L}_{MBT} + 1} = {MBT}^{{L}_{MBT}} ( {\tilde{z}}_{CMR} , {\tilde{z}}_{EHR} , {\tilde{z}}_{CIR} , {z}_{fsn} ; {θ}_{MBT} ) \qquad ( 10 )$$

$$p = sigmoid ( [ {\tilde{z}}_{CMR}^{{L}_{MBT} + 1} ] [ {\tilde{z}}_{EHR}^{{L}_{MBT} + 1} ] [ {\tilde{z}}_{CIR}^{{L}_{MBT} + 1} ] \cdot W + b ) \qquad ( 11 )$$

The training of the tri-channel reduction to practice is described presently. The data used for training the tri-channel reduction to practice were all from the first, internal, cohort. For patient i, their SCDA outcome yᵢ is 1 if they experienced a SCDA event (as defined for purposes of machine learning training herein) during the follow-up, and 0 otherwise. The model was trained to fit the paired patient multi-modality input data and their SCDA outcome, by minimizing the balanced focal loss function:

$$L = - {\sum }_{i} {{α}_{i} ( {y}_{i} - {p}_{i} )}^{γ} log {p}_{i} \qquad ( 12 )$$

In Equation (12), pᵢ=f_{tri-modality}(z_{CMR,i}, z_{EHR,i},z_{CIR,i}; θ) is output of the tri-channel reduction to practice for the i-th patient representing the predicted SCDA risk score, ai is a weighting factor assigned to each class (i.e., SCDA and no SCDA) to handle the class imbalance problem, and γ is the focusing parameter that controls the level of how the model focuses on its mistakes and prioritizes improving on the hard examples, which was set as γ=2.

The CMR, EHR and CIR sub-networks were trained individually in a first step, and then the tri-channel reduction to practice was trained end-to-end with all the sub-networks and the multi-modality fusion module. All models were trained with a batch size of 64 and a maximum of 150 epochs with early stopping based on loss. Adam optimizer was used, with β₁=0.9,β₂=0.999, and the learning rate was initially set at 1×10⁻³ for the CMR sub-network, 1×10⁻² for the EHR and CIR sub-network, 3×10⁻² for the multi-modality fusion, and was adaptively adjusted during the training process. The hyperparameters characterizing the network architecture were tuned via expert manual exploration and grid search. For the CMR sub-network, the ViT has L_{ViT}=8 transformer encoder blocks, 8 heads for each attention module, and dimension d=512. The EHR sub-network used an FNN with 2 hidden layers and a latent dimension of 16. The CIR sub-network used an FNN with 1 hidden layer and a latent dimension of 16. The extracted unimodal knowledge vectors have dimensions \tilde{z}_{CMR} ϵR³²,\tilde{z}_{EHR} ϵR¹⁶,\tilde{z}_{CIR} ϵR¹⁶. The multi-modality fusion module used a sequence of L_{MBT}=3 blocks of MBT, with a bottleneck dimension of 8, z_{fsn} ϵR⁸.

For interpretability of the LGE-MRI image sub-network, a technique called Attention Rollout was used to quantify attention flows from the start to the end throughout the ViT. Formally, at transformer encoder block l, the average of attention matrices of all attention heads is A^{l}. The residual connection at each block was modeled by adding the identity matrix l to the attention matrix. Therefore, the attention rollout may be recursively computed by:

$${A}_{Rollout}^{l} = ( {A}^{l} + I ) {A}_{Rollout}^{l - 1}$$

The image sub-network is then explainable using the Attention Rollout at the end of the ViT after flowing though L_{ViT} transformer blocks,

$${A}_{Rollout}^{{L}_{ViT}} .$$

The tri-channel reduction to practice was built in Python 3.9 using packages including PyTorch 2.0, NumPy 1.23.5, Pandas 1.5.3, SciPy 1.10, scikit-learn 1.2.0, scikit-image 0.19.3, pydicom 2.3, SimpleITK 2.2.1, and SHAP 0.41. The data preprocessing, model training, and result analysis were performed on a machine with an AMD Ryzen Threadripper 1920X 12-Core CPU and Nvidia TITAN RTX GPUs, and on the Rockfish cluster using Nvidia A100 GPU nodes, with Nvidia software CUDA 11.7 and cuDNN 8.5.

A detailed description of the performance evaluation of the tri-channel reduction to practice follows. The performance of the tri-channel reduction to practice was thoroughly evaluated using the area under the receiver operating characteristic curve (AUROC), balanced accuracy (BA), area under the precision-recall curve (AUPRC), and Brier score (Bs). In the internal 5-fold cross-validation, the tri-channel reduction to practice achieved a mean AUROC of 0.89 (95% confidence interval (CI): 0.81-0.94), a mean BA of 0.80 (95% CI: 0.71-0.88), a mean AUPRC of 0.22 (95% CI: 0.13-0.34), and a mean Bs of 0.16 (95% CI: 0.14-0.19). In the external validation, despite the dissimilarities of cohort characteristics and data missing, the tri-channel reduction to practice showed a robust prediction of SCDA events with a mean AUROC 0.81 (95%: 0.69-0.93), demonstrating its generalizability to a different healthcare-system.

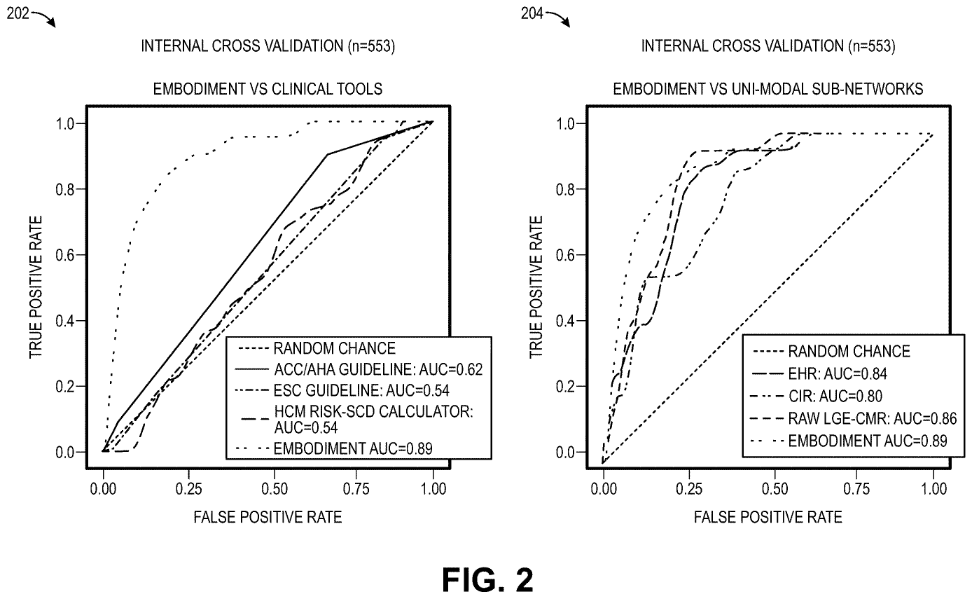

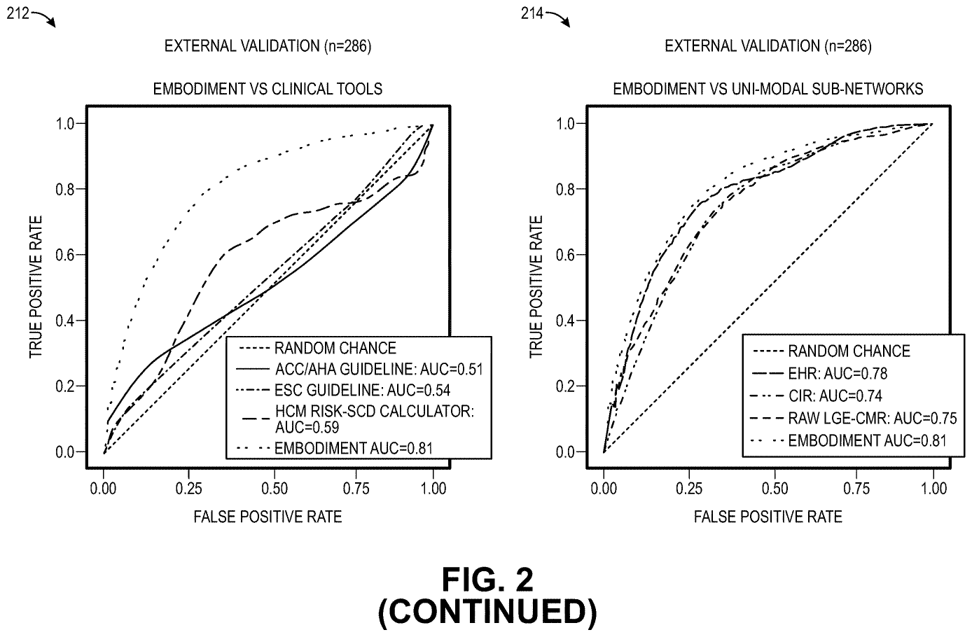

FIG. 2 depicts receiver operating characteristics curves comparing the tri-channel reduction to practice to both clinical guidelines (202, 212) and to its constituent sub-networks (204, 214), via both internal cross-validation using the first cohort (202, 204) and external validation using the second cohort (212, 214).

As shown in FIG. 2, the clinical guidelines offered limited SCDA prediction accuracy. In the internal cross-validation, the ACC/AHA guideline listed 7 major SCD risk factors ( 6/7 were included in this analysis) and had a mean AUROC of 0.62 (95% (CI): 0.51-0.72). The ESC guideline listed 7 major SCD risk factors (all included) and had a mean AUROC of 0.54 (95% CI: 0.42-0.66). The HCM Risk-SCD calculator used 7 risk factors to calculate a 5-year SCD risk score, resulting in a mean AUROC of 0.54 (95% CI: 0.42-0.64). In contrast, the tri-channel reduction to practice offered improvements of 0.27-0.35 in AUROC. If the tri-channel reduction to practice were chained to have the same level of sensitivity as the clinical guidelines, the tri-channel reduction to practice offered improvements of 0.33-0.5 in specificity. In the external validation, the clinical tools could hardly predict SCDA events again, with AUROC of 0.51-0.59. The tri-channel reduction to practice offered improvements of 0.22-0.30 in AUROC.

As further shown in FIG. 2, the performance of the tri-channel reduction to practice was evaluated relative to the unimodality sub-networks within the tri-channel reduction to practice, when they operated independently. In the internal validation, among the three unimodality sub-networks, the image-only sub-network had the highest AUROC (mean: 0.86; 95% CI: 0.80-0.91), and the CIR-only sub-network had the lowest AUROC (mean: 0.80, 95 CI: 0.72-0.87). Despite that CIR data already included manually crafted measurements from LGE-MRI images (e.g. LGE (%), LV scar tissue mass), the CMR-only sub-network still outperformed the CIR-only sub-network, demonstrating that the neural network learned additional information from the raw LGE-MRI images which were not fully accounted for in standard radiology reports. The multi-modality AI's AUROC performance edge over the unimodality sub-networks was 0.03-0.09, and the differences were tested to be statistically significant in paired-samples t-tests (p-value<0.001). In the external validation, the tri-channel reduction to practice still outperformed all the unimodality sub-networks, with AUROC performance edge of 0.03-0.07. Although the image-only sub-network had a relatively high performance drop of 0.11 in AUROC, compared with 0.06 AUROC drops in EHR- and CIR-only sub-networks, the tri-channel reduction to practice managed to keep a relatively low performance drop of 0.07 in AUROC.

The tri-channel reduction to practice, as well as the individual unimodality models, far surpassed the existing clinical tools in stratifying SCDA risk. Each offered discriminative risk scores for stratifying SCDA risks, and the risk score distributions between patients who experienced SCDA and those who didn't experience SCDA were tested to be significantly different in two-sample K-S tests (p-value<0.001). By comparison, none of the clinical tools were able to stratify SCDA risk effectively (p-value>0.05).

An evaluation of the effects of adding data modalities is set forth presently. The EHR data reflect the baseline information about a patient, and the EHR-only sub-network (FNN) could achieve a mean AUROC of 0.84, mean BA of 0.77, mean AUPRC of 0.16, and mean Bs of 0.18 in the internal cross-validation. A comparison in sequence of the EHR-only FNN (AUROC=0.84), a reduction to practice using EHR and CIR (AUROC=0.87), and the tri-channel reduction to practice using all three data types (of two different modalities) (AUROC=0.89) indicated stepwise enhancements in all performance metrics, and no evident difference in their CI ranges, suggesting that integrating multiple aspects of assessments in a risk stratification model reduces the bias without compromises of variance.

In addition, to elucidate the utility of the multi-modality fusion module within the tri-channel reduction to practice, results from a control model were compared. The control model is an FNN taking the concatenation of EHR data and CIR data in the input directly, which is an example of an early-fusion paradigm. Instead, the tri-channel reduction to practice adopts a mid-fusion paradigm, with the idea of having the first dedicated layers learn modality-specific knowledge from separate inputs, and fusing the distilled multi-modal knowledge in the middle of the network. Results showed that the control model had lower performance (AUROC=0.81) than the reduction to practice using EHR and CIR, and it had even lower performance than the EHR-only sub-network, despite more data modalities available to it. The tri-channel reduction to practice had superior performance to all of the evaluated systems. The low performance of the control model might be due to overfitting; however, the multi-modal design within the tri-channel reduction to practice attenuates the overfitting caused by adding data modalities.

An evaluation of the tri-channel reduction to practice's performance in different sex- and age-subgroups of the JHH-HCM cohort in the cross-validation is set forth presently. The tri-channel reduction to practice far surpassed the clinical tools in all the subgroups. In the sex-subgroup evaluations, the tri-channel reduction to practice had AUROC improvements of 0.26-0.36, and had BA improvements of 0.15-0.29. In the age-subgroup evaluations, the tri-channel reduction to practice had even larger improvements: 0.21-0.46 improvements in AUROC and 0.16-0.36 improvements in BA.

The tri-channel reduction to practice also exhibited inter-subgroup stability. For different sex-subgroups, the tri-channel reduction to practice had AUROC variability of 0.02 and BA variability of 0.05. In general, the performance stability of the tri-channel reduction to practice demonstrated its fairness for different demographic subgroups.

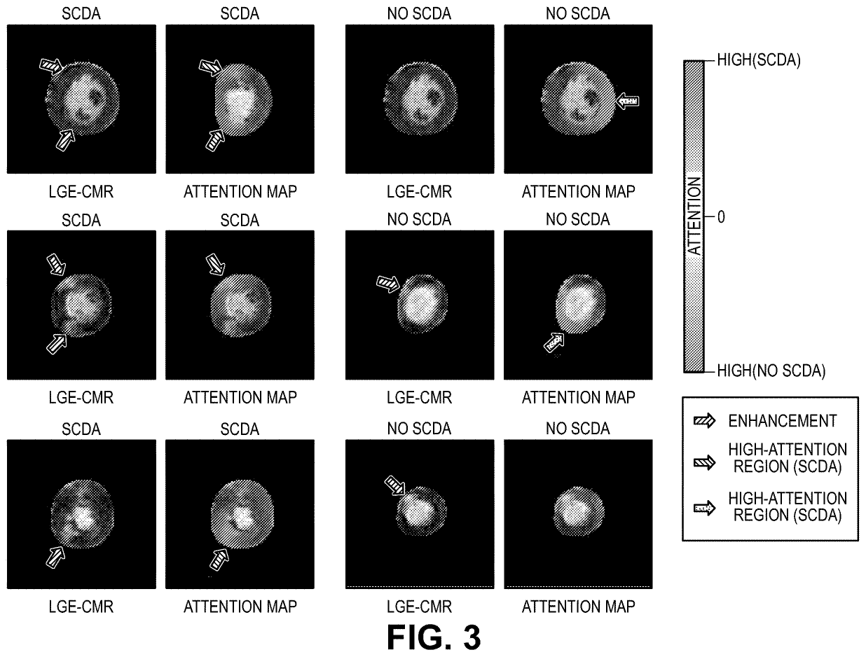

A discussion of interpretability provided by of the tri-channel reduction to practice is set forth presently in reference to FIG. 3.

FIG. 3 illustrates interpretability of the tri-channel reduction to practice based on attention to various regions of LGE-MRI images. Attribution- and attention-based interpretation was applied to the tri-channel reduction to practice in order to explain how EHR features, CIR measurements, and raw LGE-MRI images were used to predict SCDA risks. The 3D ViT sub-network used for LGE-MRI image analysis has innate attention mechanisms that allow for interpretation of the knowledge it learned from the images. Attention flows in the trained ViT sub-network were quantified using attention rollout, and the attention to input images are presented as heatmaps that show relative associations between image regions and prognostic SCDA risks. As shown in FIG. 3, the tri-channel reduction to practice does not always pay high attention to LGE regions, meaning that it does not serve as a simple LGE identifier that uses total LGE burden to estimate SCDA risk. In LGE-MRI images from patients who experienced SCDA, the regions that the tri-channel reduction to practice pays high attention to tend to overlap with LGE regions. In contrast, for patients who did not experience SCDA, the tri-channel reduction to practice tends to distract from the LGE regions of their respective image. It suggests that the tri-channel reduction to practice has learned a nuanced relationship between LGE and the propensity for SCDA.

### III. Bi-Channel Reduction to Practice

This section presents a reduction to practice that predicts and interprets the risk of arrhythmic sudden cardiac death (SCDA) in patients using bi-modality medical data and evaluated in HCM patient cohorts. The bi-channel reduction to practice employs a neural network system to analyze data from electric health records (EHR), including echocardiogram and LGE-MRI (CMR) reports, as well as raw LGE-MRI images, which reveal disease-induced myocardial fibrosis. The bi-channel reduction to practice includes two unimodality sub-networks, or paths, for extracting modality-specific knowledge, as well as a multi-modality fusion component to fuse knowledge and distill holistic representations for prognostic SCDA risk stratification.

FIG. 4 is a schematic diagram of a bi-channel machine learning system 400 for ventricular arrhythmia risk prediction for a hypertrophic cardiomyopathy patient, according to a second reduction to practice. The system 400 has two dedicated unimodality sub-network branches designed for each of two different inputs, namely, raw LGE-MRI images and clinical covariates from EHR, the latter of which including measurements from cardiac imaging reports (CIR). The image branch included a 3D ResNeXt neural network, which extracts features from processed LGE-MRI images. The EHR data branch included a multi-layer perceptron, which extracts features from processed clinical covariates. The multi-modal fusion component included a cross-attention transformer to synergistically fuse knowledge from both branches for VA risk stratification.

The training data for the bi-channel reduction to practice included data for 819 individuals. For each individual, the training data included a respective LGE-MRI image and respective EHR data. The EHR data included demographics data (e.g., age, race, gender, height, etc.), medical records data (e.g., medications such as diuretics, disease history, family history, etc.), echocardiogram data (e.g., ejection fraction, IVS thickness, strain rates, etc.), and LGE-MRI data (e.g., LV mass, LGE mass, LGE volume, etc.). The labels for the training data were indicative of the target outcomes, namely, the event type (SCDA or non-SCDA), where SCDA was indicated by sustained ventricular tachycardia or ventricular fibrillation. For the bi-channel reduction to practice, the patient cohort for the training data had a mean follow-up duration of 2.86 years; therefore, the output risk score reflected a patient's risk for VA in this timeframe.

FIG. 5 depicts receiver operating characteristics curves comparing the bi-channel reduction to practice to both clinical guidelines (502) and to its constituent sub-networks (504). As shown in FIG. 5, the bi-channel reduction to practice demonstrated that the method using either LGE-MRI (AUC=0.84) or EHR (AUC=0.86) alone, i.e., using only one sub-network, outperformed the latest clinical criteria outlined by ACC/AHA (AUC=0.71) or ESC (AUC=0.69) in predicting VA risk. When provided with both data, the bi-channel reduction to practice achieved even higher performance (AUC=0.91).

The bi-channel reduction to practice also maintained excellent performance in different subgroups of patients, including gender, age, and race. Its robustness suggests that embodiments can provide accurate risk stratification for a wide range of HCM patients, regardless of demographic or clinical factors. Moreover, the interpretability of the bi-channel reduction to practice allows for identification of specific regions from raw LGE-MRI images and covariates from EHR that contributed to the personalized risk score for VA. With the accuracy, fairness, and transparency in identifying patients at high risk of VA, embodiments are expected to help improve HCM patient care and guide life-saving treatment to those who need it most.

### IV. Conclusion

Example commonalities between the tri-channel reduction to practice and the bi-channel reduction to practice are described presently, by way of non-limiting examples of some embodiments. In general, the reductions to practice may be conceptualized as including three stages, as follows.
1. Data processing protocols, e.g., to clean and prepare LGE-MRI image, EHR, and CIR data,
2. Scalable multi-modal deep learning modeling to predict SCDA risk from patient data; and
3. Interpretation of the trained machine learning system's output to derive clinical insights.

In the first stage, for LGE-MRI processing, a deep learning approach is used for left ventricle myocardium segmentation, which includes a cascade of three neural networks and a mathematical anatomy-check component. Further, the LGE-MRI image intensity and spacing is standardized. For EHR processing, EHR covariates with high percentages of missing values (e.g., >40%) are excluded. A protocol is used for cleaning EHR data and imputing the missing values of the remaining covariates.

The second stage uses a novel multi-modal machine learning system, with dedicated parallel branches for at least LGE-MRI images inputs and certain EHR inputs, and possibly with a third parallel branch for CIR inputs, as well as a multi-modality fusion module, to predict personalized patient SCDA risk. The LGE-MRI branch was implemented using a 3D ResNeXt neural network for the bi-channel reduction to practice and using a 3D vision transformer for the tri-channel reduction to practice, by way of non-limiting examples. The EHR branch was implemented using a multi-layer perceptron for the bi-channel reduction to practice and using a feedforward neural network for the tri-channel reduction to practice, by way of non-limiting examples. The CIR branch was implemented using a feedforward neural network for the tri-channel reduction to practice, by way of non-limiting example. The multi-modal fusion component was implemented using a cross-attention transformer for the bi-channel reduction to practice and using a multi-modal bottleneck transformer for the tri-channel reduction to practice, byway of non-limiting examples.

For the third stage, the trained machine learning model may be interpreted to identify relevant covariates and tissues to SCDA propensity. Shapley values may be calculated for each clinical covariate to represent its propensity for SCDA. Attributions of each region in the LGE-MRI images may be calculated using the Occlusion method. And clinical implications of the identified covariates and regions may be analyzed.

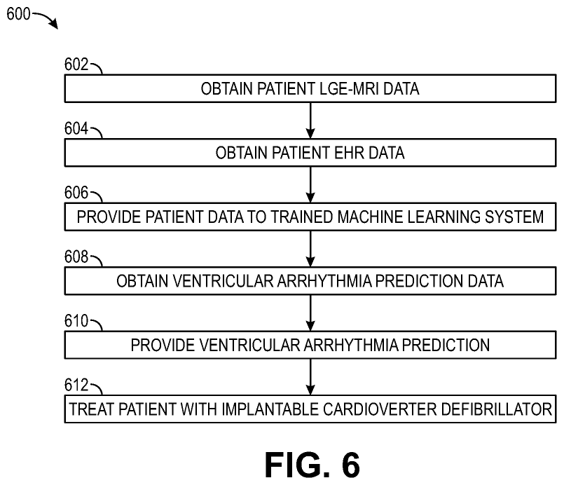

FIG. 6 is a flow diagram for a machine learning method 600 for ventricular arrhythmia risk prediction for a hypertrophic cardiomyopathy patient, according to various embodiments. The method 600 may be practiced using a trained machine learning system, such as are shown and described herein in reference to FIGS. 1-5.

At 602, the method 600 includes obtaining a patient LGE-MRI image of at least the patient's left ventricle. The LGE-MRI image may be obtained directly from an MRI system, from electronic persistent memory, or over a network, by way of non-limiting examples.

At 602, the method 600 includes obtaining patient electronic health records data of the patient. The electronic health records data may be obtained from electronic persistent memory, such as from an electronic health records system, and/or over a network, by way of non-limiting examples. According to some embodiments, the electronic health records data may include patient demographics data and patient medical records data. According to some embodiments, the electronic health records data may include any, or any combination, of data as represented in Table 1. According to some embodiments, the electronic health records data may include patient cardiac imaging report data. According to some embodiments, the cardiac imaging report data may include any, or any combination, of data as represented in Table 2.

At 606, the method 600 includes providing patient data to a trained machine learning system, where the patient data includes the patient LGE-MRI image and the patient electronic health records data. The trained machine learning system may be as shown and described herein in reference to FIGS. 1 and/or 4, for example. The trained machine learning system may include: an image branch trained using a training corpus of LGE-MRI images, an electronic health records data branch trained using a training corpus of electronic health records data, and a multi-modality fusion component. Inputs to the multi-modality fusion component may be coupled to at least an output of the image branch and to and output of the electronic health records data branch. The patient LGE-MRI image may be input to the image branch, and at least some of the patient electronic health records data may be input to the electronic health records data branch.

According to some embodiments, e.g., as shown and described herein in reference to FIG. 1, the patient electronic health records data may include patient cardiac imaging report data of the patient. According to such embodiments, the trained machine learning system may further include a cardiac imaging report data branch trained using a training corpus of cardiac imaging report data, and the cardiac imaging report data may be input to the cardiac imaging report data branch. According to such embodiments, an input to the multi-modality fusion component may be coupled to an output of the cardiac imaging report data branch.

At 608, the method 600 includes obtaining, from the trained machine learning system, ventricular arrhythmia prediction data. The ventricular arrhythmia prediction data may include a probability or other assessment of sudden cardiac death due to arrhythmia during a specified timeframe. The ventricular arrhythmia prediction data may be output from the trained machine learning system, e.g., output from the multi-modality fusion component.

At 610, the method 600 includes providing a ventricular arrhythmia prediction based on the ventricular arrhythmia prediction data. The ventricular arrhythmia prediction may be in the form of a probability or other assessment of sudden cardiac death due to arrhythmia during a specified timeframe. According to some embodiments, the ventricular arrhythmia prediction is numeric, e.g., a probability. According to some embodiments, the ventricular arrhythmia prediction is categorical, such as one of “low,” “medium,” or “high,” e.g., as ascribed to predetermined intervals of numeric ventricular data. The ventricular arrhythmia prediction may be provided by display on a monitor, transmission over a network, or storage in persistent memory, e.g., in a medical records system, by way of non-limiting examples.

At 612, the method 600 includes treating the patient with an implantable cardioverter defibrillator, e.g., when the ventricular arrhythmia prediction represents a sufficiently high risk. By way of non-limiting example, the patient may be treated with an implantable cardioverter defibrillator if the ventricular arrhythmia prediction exceeds a predetermined threshold for a numeric ventricular arrhythmia prediction, or if the ventricular arrhythmia prediction is in a specified category, e.g., “high.”

Multi-modality fusion according to various embodiments has many advantages over previous techniques. For example, embodiments extract features from raw LGE-MRI images that are not accounted for in standard radiology reports. Further, the reductions to practice out-perform the latest clinical standard in the risk stratification for VA, having higher accuracy and AUROC and demonstrating fairness when evaluated on different subgroups of patients. Yet further, the interpretability of the reductions to practice not only allow for identifying important risk factors for VA, but also improves the transparency of the personalized risk predictions.

Additional advantages of various embodiments derive at least in part from the “mid-fusion” paradigm as implemented by the reductions to practice disclosed herein. In contrast to techniques that simply build models based on a bag of all features, e.g., processed by a single channel, and ignoring the interplay between different data modalities, some embodiments utilize a unified multi-modal framework with a dedicated fusion component that specially shares knowledge across modalities and learns the cross-modality interplay. The reductions to practice yielded superior performance than any unimodality sub-network and also reduced performance variability in subgroup evaluations. Moreover, the high performance of the reductions to practice is due not only to added data modalities, but also to the effective integration of multi-modal knowledge. For example, the reductions to practice outperform the early-fusion paradigm in SCDA risk prediction, despite the fact that they use similar data modalities.

Regarding the advantageous interpretability of the reductions to practice, the analysis may be performed on the group or individual level, allowing for studying the global effect of any feature at the group level, or explaining how the model makes its prediction for a particular patient at the individual level. This multi-level interpretability is usable not only for clinicians to more transparently understand an embodiment as a clinical decision support tool in SCDA risk stratification, but also for the broader biomedical research community to investigate the discoveries behind the interpretations.

Many variations and modifications are possible. For example, some embodiments may utilize modalities different from, or in addition to, raw LGE-MRI images and clinical covariates from EHR. Such alternative or additional modalities include, by way of non-limiting example, times series data (e.g., ECG). Note that time series data represents a different modality from image data and numeric/Boolean data modalities.

Certain examples can be performed using a computer program or set of programs. The computer programs can exist in a variety of forms both active and inactive. For example, the computer programs can exist as software program(s) comprised of program instructions in source code, object code, executable code or other formats, firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by an electronic processor.

These computer readable program instructions may be provided to a processor of a general-purpose computer, special purpose computer, or other programmable data processing apparatus to produce a machine, such that the instructions, which execute via the electronic processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including Smalltalk, C++, Python, C, any object oriented programming language, and/or any procedural programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A machine learning method for ventricular arrhythmia risk prediction for a hypertrophic cardiomyopathy patient, the method comprising:
obtaining a patient late gadolinium enhancement cardiac MRI (LGE-MRI) image of at least the patient's left ventricle;
obtaining patient electronic health records data of the patient;
providing patient data to a trained machine learning system, wherein the patient data comprises the patient LGE-MRI image and the patient electronic health records data, wherein the trained machine learning system comprises:
an image branch trained using a training corpus of LGE-MRI images,
an electronic health records data branch trained using a training corpus of electronic health records data, and
a multi-modality fusion component, wherein the multi-modality fusion component is coupled to at least the image branch and the electronic health records data branch, and
wherein the providing comprises inputting the patient LGE-MRI image to the image branch and inputting at least some of the patient electronic health records data to the electronic health records data branch;
obtaining, from the trained machine learning system, ventricular arrhythmia prediction data; and
providing a ventricular arrhythmia prediction based on the ventricular arrhythmia prediction data.

2. The method of claim 1, further comprising:
determining that the ventricular arrhythmia prediction exceeds a predetermined threshold for a numeric ventricular arrhythmia prediction, or
determining that the ventricular arrhythmia prediction is in a predetermined category for a categorical arrhythmia prediction; and
treating the patient with an implantable cardioverter defibrillator in response to the determining.

3. The method of claim 1,
wherein the patient electronic health records data comprises patient cardiac imaging report data of the patient,
wherein the trained machine learning system further comprises a cardiac imaging report data branch trained using a training corpus of cardiac imaging report data,
wherein the providing further comprises inputting the cardiac imaging report data to the cardiac imaging report data branch, and
wherein the multi-modality fusion component is further coupled to the cardiac imaging report data branch.

4. The method of claim 3,
wherein the patient electronic health records data comprises: patient demographics data and patient medical records data, and
wherein the patient cardiac imaging report data comprises patient echocardiogram data and patient cardiac MRI data.

5. The method of claim 1, wherein the patient electronic health records data comprises: patient demographics data, patient medical records data, patient echocardiogram data, and patient cardiac MRI data.

6. The method of claim 1, further comprising automatically identifying a region of interest that tightly contains a left ventricular myocardium of the patient LGE-MRI image using a neural network.

7. The method of claim 1, wherein the ventricular arrhythmia prediction data represents a probability of sudden cardiac death due to arrhythmia during a specified timeframe.

8. The method of claim 1, wherein the image branch comprises a three dimensional vision transformer.

9. The method of claim 1, wherein the image branch comprises a first neural network, and wherein the electronic health records branch comprises a second neural network.

10. The method of claim 1, wherein the multi-modality fusion branch comprises a transformer.

11. A machine learning system for ventricular arrhythmia risk prediction for a hypertrophic cardiomyopathy patient, the system comprising: a non-transitory computer readable medium comprising instructions; and at least one electronic processor that executes the instructions to perform operations comprising:
obtaining a patient late gadolinium enhancement cardiac MRI (LGE-MRI) image of at least the patient's left ventricle;
obtaining patient electronic health records data of the patient;
providing patient data to a trained machine learning system, wherein the patient data comprises the patient LGE-MRI image and the patient electronic health records data, wherein the trained machine learning system comprises:
an image branch trained using a training corpus of LGE-MRI images,
an electronic health records data branch trained using a training corpus of electronic health records data, and
a multi-modality fusion component, wherein the multi-modality fusion component is coupled to at least the image branch and the electronic health records data branch, and
wherein the providing comprises inputting the patient LGE-MRI image to the image branch and inputting at least some of the patient electronic health records data to the electronic health records data branch;
obtaining, from the trained machine learning system, ventricular arrhythmia prediction data; and
providing a ventricular arrhythmia prediction based on the ventricular arrhythmia prediction data.

12. The system of claim 11, wherein the operations further comprise:
determining that the ventricular arrhythmia prediction exceeds a predetermined threshold for a numeric ventricular arrhythmia prediction, or
determining that the ventricular arrhythmia prediction is in a predetermined category for a categorical arrhythmia prediction,
wherein the patient is treated with an implantable cardioverter defibrillator in response to the determining.

13. The system of claim 11,
wherein the patient electronic health records data comprises patient cardiac imaging report data of the patient,
wherein the trained machine learning system further comprises a cardiac imaging report data branch trained using a training corpus of cardiac imaging report data,
wherein the providing further comprises inputting the cardiac imaging report data to the cardiac imaging report data branch, and
wherein the multi-modality fusion component is further coupled to the cardiac imaging report data branch.

14. The system of claim 13,
wherein the patient electronic health records data comprises: patient demographics data and patient medical records data, and
wherein the patient cardiac imaging report data comprises patient echocardiogram data and patient cardiac MRI data.

15. The system of claim 11, wherein the patient electronic health records data comprises: patient demographics data, patient medical records data, patient echocardiogram data, and patient cardiac MRI data.

16. The system of claim 11, wherein the operations further comprise automatically identifying a region of interest that tightly contains a left ventricular myocardium of the patient LGE-MRI image using a neural network.

17. The system of claim 11, wherein the ventricular arrhythmia prediction data represents a probability of sudden cardiac death due to arrhythmia during a specified timeframe.

18. The system of claim 11, wherein the image branch comprises a three dimensional vision transformer.

19. The system of claim 11, wherein the image branch comprises a first neural network, and wherein the electronic health records branch comprises a second neural network.

20. The system of claim 11, wherein the multi-modality fusion branch comprises a transformer.
